(19) United States
(12) Patent Application Publication
(10) Pub. No.: US 2025/0367415 A1
(43) Pub. Date: Dec. 4, 2025

(54) ROLLING TOROIDAL BALLOON CATHETER SYSTEMS WITH OPTIONAL MULTI-BALLOON CONFIGURATIONS, INTERNAL FRICTION REDUCTION, LOW-IMPACT REMOVAL, AND SAFETY RELEASE MECHANISMS

(71) Applicant: Proluma Medical, Inc., Boston, MA (US)

(72) Inventors: J. Mathieu Massicotte, North Reading, MA (US); Peter J. Massicott, Boston, MA (US); Troy Ziegler, Apple Valley, MN (US)

(73) Assignee: Proluma Medical, Inc., Boston, MA (US)

(21) Appl. No.: 19/223,687

(22) Filed: May 30, 2025

Related U.S. Application Data
(60) Provisional application No. 63/653,289, filed on May 30, 2024.

(51) Int. Cl. A61M 25/10; A61M 25/00

(52) U.S. Cl. CPC A61M 25/1011; A61M 25/0017; A61M 25/0045; A61M 25/1002; A61M 25/10185; A61M 2025/0046; A61M 2025/1013; A61M 2025/1088; A61M 2205/0216; A61M 2205/0222; A61M 2205/0238; A61M 2205/3331; A61M 2210/1085

(57) ABSTRACT

An improved catheter has an elongated shaft with at least one drainage lumen and one or more toroidal balloons coaxially mounted thereto. Each balloon provides an internal balloon surface that is either continuous, forming an inner channel and a first toroidal space, or non-continuous, the surface and shaft bounding a second toroidal space at attachment points. Lubricant applied to the shaft and/or the toroidal spaces lowers friction where opposed portions of the internal surface meet or contact the shaft, enabling rolling or inversion of the balloon while its external surface remains stationary against the lumen wall. Optional features include dedicated inflation ports, inflation/deflation valves with pressure-relief elements, rupture seams actuated by a cutting ring, retention balloons, and spaced toroidal balloons positioned to shield the prostate and distal urethra.

### CROSS-REFERENCE TO RELATED APPLICATIONS

This application claims benefit of U.S. Provisional Application No. 63/653,289, filed May 30, 2024, which is hereby incorporated herein by reference in its entirety.

### TECHNICAL FIELD

The present invention relates generally to medical devices. More specifically, the present invention involves rolling toroidal balloon catheter systems with optional multi-balloon configurations, internal friction reduction, low-impact removal, and safety release mechanisms.

### BACKGROUND OF THE INVENTION

Conventional medical balloons are most often spherical, cylindrical, or sausage-shaped. They are introduced into the body in a deflated state, inflated to perform a therapeutic or anchoring function, and then deflated again for removal. Because their inflated geometry is essentially fixed, such balloons do not accommodate significant relative motion between the balloon and the medical device on which they are mounted. Toroidal, or “donut-shaped,” balloons are also known, for example, as inflatable cuffs on endotracheal tubes. Although a toroidal balloon provides a patent central channel when inflated, it is typically used to dilate an anatomical passage or to hold a device in place, rather than to facilitate controlled movement of a device along or through that channel. In existing designs, therefore, the balloon's purpose remains static even when the balloon is toroidal, and the ability to use the balloon itself to enable low-friction translation of a catheter or other instrument has not been realized.

Subsequent developments introduced rolling toroidal balloons, balloons that, while inflated, can translate axially by inverting on themselves, thereby offering the potential for greater functional versatility and reduced tissue trauma. Such rolling-toroid technology was first disclosed by J. M. Massicotte and P. J. Massicotte in U.S. Pat. No. 8,343,170 (filed Aug. 14, 2006) and later refined in U.S. Pat. No. 8,529,581 (filed Dec. 4, 2012). A medical application of that concept on a urinary-drainage device, a modified Foley catheter, was subsequently described in U.S. Pat. No. 10,213,208 (filed Mar. 23, 2015). As commercial development of the toroidal Foley catheter has progressed, additional design variations have emerged that address safety, manufacturability, and clinical usability. The present disclosure sets out those novel modifications and seeks corresponding protection.

Embodiments of the present invention are an improvement over prior art systems and methods.

### SUMMARY OF THE INVENTION

In one embodiment, the present invention provides a catheter comprising: (a) an elongated catheter shaft defining at least one drainage lumen; (b) at least one toroidal balloon disposed coaxially around the catheter shaft and having an internal balloon surface, the internal balloon surface being either: (i) continuous across the catheter shaft, thereby defining an inner channel through which the catheter shaft extends, the internal balloon surface enclosing a first toroidal space; or (ii) non-continuous across the catheter shaft, the internal balloon surface having circumferential first and second attachment points to the catheter shaft, such that the internal balloon surface together with the catheter shaft bounds a second toroidal space; (c) an external balloon surface configured, in use, to contact a body lumen wall; and (d) a lubricant selectively applied to (i) the catheter shaft, (ii) the internal balloon surface, (iii) the first toroidal space when the internal balloon surface is continuous, or (iv) the second toroidal space when the internal balloon surface is non-continuous, the lubricant reducing friction at regions where opposed portions of the internal balloon surface contact each other and at regions where the internal balloon surface contacts the catheter shaft, thereby allowing smooth relative movement of the toroidal balloon while substantially limiting motion of the external balloon surface against surrounding tissue.

In one embodiment, in addition to the second toroidal space, the internal balloon surfaces form a third toroidal space circumferentially adjacent to the first attachment point; and the internal balloon surfaces form a fourth toroidal space circumferentially adjacent to the second attachment point.

In one embodiment, the lubricant is selected from the group consisting of a lubricious coating such as a hydrophilic coating, a liquid lubricant, a dry lubricant, an inherently lubricious material, an embedded fabric, a textured surface molded directly into the balloon surface, and combinations thereof.

In one embodiment, the catheter shaft is translatable along its longitudinal axis relative to the toroidal balloon, such that, during catheter withdrawal, at least a portion of the toroidal balloon inverts, thereby reducing friction between the catheter and the body lumen wall.

In one embodiment, the catheter further comprises a pressure-control system, the pressure-control system comprising: (a) an inflation/deflation valve fluidly coupled to the toroidal balloon, and (b) a relief valve mounted on the wall of the toroidal balloon and physically separate from the inflation/deflation valve, wherein the relief valve is configured to vent the balloon at a preset pressure.

In one embodiment, the inflation/deflation valve further includes an integral pressure-relief element in addition to the relief valve.

In one embodiment, the toroidal balloon includes an inflation port disposed directly on the balloon wall at a location spaced from the balloon's attachment region to the catheter shaft.

In one embodiment, the toroidal balloon is configured, upon reaching a rolling limit, to provide a retention function that anchors the catheter in a bladder in the absence of a separate retention balloon.

In one embodiment, the toroidal balloon includes a rupture feature selected from an internal breakaway seam, an external serrated tear line, or a zip thread.

In one embodiment, the rupture feature is actuated by a ring carrying a cutting surface positioned to sever the toroidal balloon when the balloon reaches a predetermined rotational position.

In one embodiment, the catheter further comprises a retention balloon, wherein the toroidal balloon overlaps a portion of the retention balloon when both are inflated. In one embodiment, the toroidal balloon is inserted in a pre-inflated state.

In one embodiment, the toroidal balloon is positioned along the catheter tube to oppose a predetermined anatomical region selected from the group consisting of the prostate, a surgical site associated with the prostate, and the distal urethra.

In one embodiment, the catheter further comprises a retention balloon disposed coaxially around the catheter shaft, wherein the retention balloon is configured to inflate into a toroidal shape and is secured to the catheter shaft by closely spaced circumferential attachment points forming a narrow neck or attachment band, thereby permitting axial sliding or rolling motion of the retention balloon longitudinally along the catheter shaft.

In one embodiment, the catheter further comprises a retention balloon coaxially mounted around the catheter shaft, wherein the retention balloon is secured to the catheter shaft by a narrow attachment point or neck, and wherein the retention balloon is formed from a soft, flexible material configured to roll bidirectionally inward toward, and outward away from, the attachment point when axial force is applied to the catheter shaft, thereby facilitating axial motion of the catheter shaft and reducing shear forces and tissue trauma.

In another embodiment, the present invention provides a catheter comprising: (a) an elongated catheter shaft defining at least one drainage lumen; (b) first and second toroidal balloons disposed coaxially around the catheter shaft and axially spaced apart from one another, each of the first and second toroidal balloons including: (i) an internal balloon surface that is either: (1) continuous across the catheter shaft, thereby defining an inner channel through which the catheter shaft extends and the internal balloon surface encloses a first toroidal space; or (2) non-continuous across the catheter shaft, the internal balloon surface having circumferential first and second attachment points to the catheter shaft, such that the internal balloon surface together with the catheter shaft bounds a second toroidal space; (ii) an external balloon surface configured, in use, to contact a body lumen wall; and (iii) a lubricant selectively applied to (A) the catheter shaft, (B) the internal balloon surface, (C) the first toroidal space when the internal balloon surface is continuous, or (D) the second toroidal space when the internal balloon surface is non-continuous, the lubricant reducing friction at regions where opposed portions of the internal balloon surface contact each other and at regions where the internal balloon surface contacts the catheter shaft, thereby allowing smooth relative movement of each toroidal balloon while substantially limiting motion of the external balloon surface against surrounding tissue.

In one embodiment, the catheter further comprises a pressure-control system comprising at least one inflation valve fluidly coupled to the toroidal balloons and at least one pressure-relief valve associated with at least one of the toroidal balloons:

In one embodiment, the first toroidal balloon and the second toroidal balloon each have respective dedicated inflation ports.

In one embodiment, either the first toroidal balloon or second toroidal balloon includes its own dedicated inflation port.

In one embodiment, the first toroidal balloon is positioned to oppose a patient's prostate or a surgical site near a patient's prostate and the second toroidal balloon is positioned to oppose a distal urethral segment.

In one embodiment, the second toroidal balloon is configured, upon reaching a rolling limit, to provide a retention function that anchors the catheter in a bladder in the absence of a separate retention balloon.

In one embodiment, the at least one of the toroidal balloons includes a rupture feature selected from an internal breakaway seam, an external serrated tear line, or a zip thread.

In one embodiment, the rupture feature is actuated by a ring carrying a cutting surface positioned to sever the toroidal balloon when that balloon reaches a predetermined rotational position.

In one embodiment, the catheter further comprises a retention balloon, wherein at least a portion of at least one of the toroidal balloons overlaps a portion of the retention balloon or a portion of the retention balloon overlaps a portion of at least one of the toroidal balloons when both are inflated.

In one embodiment, the toroidal balloons are inserted in a pre-inflated state.

In one embodiment, at least one pressure-relief valve is mounted directly on the wall of one of the toroidal balloons.

### BRIEF DESCRIPTION OF FIGURES

The present disclosure, in accordance with one or more various examples, is described in detail with reference to the following figures. The drawings are provided for purposes of illustration only and merely depict examples of the disclosure. These drawings are provided to facilitate the reader's understanding of the disclosure and should not be considered limiting of the breadth, scope, or applicability of the disclosure. It should be noted that for clarity and ease of illustration these drawings are not necessarily made to scale.

FIGS. 1A and 1B depict a prior-art toroidal Foley catheter, with the toroidal balloon shown deflated in FIG. 1A and inflated in FIG. 1B.

FIGS. 2A and 2B depict alternative pressure-relief arrangements: FIG. 2A shows the relief valve integrated into the balloon's inflation valve, whereas FIG. 2B shows the relief valve mounted separately on the toroidal-balloon wall.

FIGS. 3A and 3B depict two inflation-port locations, with FIG. 3A illustrating a balloon inflated at its catheter-attachment site (prior art) and FIG. 3B illustrating a balloon inflated through a valve positioned elsewhere on the balloon wall.

FIGS. 4A-4C depict a catheter carrying two axially spaced toroidal balloons. FIG. 4A feeds both balloons from a common inflation port; FIG. 4B provides each balloon with a dedicated port, and FIG. 4C combines a common port with an additional balloon-specific port.

FIG. 5 depicts a toroidal-balloon construction in which the top and/or bottom balloon portions can overlap a retention balloon.

FIG. 6 depicts a stand-alone toroidal-balloon sleeve that includes its own on-balloon inflation port and can be slipped over an off-the-shelf catheter.

FIG. 7 depicts a catheter lacking a separate retention bulb, in which a single toroidal balloon anchors the catheter after reaching its rolling limit.

FIGS. 8A-8E depict the inversion sequence of the toroidal balloon during withdrawal and, in FIGS. 8D-8E, a ring-and-cutting surface mechanism that cuts the balloon when rotation stops to convert it into a sliding sleeve.

FIGS. 9A-9C depict a retention balloon attached by a narrow neck that allows the balloon to translate axially along the catheter shaft.

FIGS. 10A-10C depict a retention balloon designed to roll inward and outward over its attachment point in response to bidirectional axial loads.

FIG. 11 depicts a retention balloon whose primary inflation valve incorporates an over-pressure relief function for automatic deflation under excessive traction.

FIGS. 12A and 12B depict retention-balloon safety variants, with FIG. 12A adding a separate pressure-relief valve to the main valve, and FIG. 12B employing a balloon wall engineered to burst and deflate at a preset pressure.

FIG. 13 depicts a flexible non-balloon retention device that folds flat for insertion, opens in the bladder, and folds forward into the toroidal channel on forced extraction.

FIGS. 14A and 14B depict an alternative folding retention element that opens inside the bladder and is enveloped by the inverting toroidal balloon during catheter withdrawal.

FIGS. 15A-C depict alternate embodiments illustrating various configurations of a toroidal balloon attached to a catheter shaft.

### DETAILED DESCRIPTION

While this invention is illustrated and described in a preferred embodiment, the invention may be produced in many different configurations. There is depicted in the drawings, and will herein be described in detail, a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the present invention.

Note that in this description, references to “one embodiment” or “an embodiment” mean that the feature being referred to is included in at least one embodiment of the invention. Further, separate references to “one embodiment” in this description do not necessarily refer to the same embodiment; however, neither are such embodiments mutually exclusive, unless so stated and except as will be readily apparent to those of ordinary skill in the art. Thus, the present invention can include any variety of combinations and/or integrations of the embodiments described herein.

The various embodiments described herein are novel improvements over the prior art.

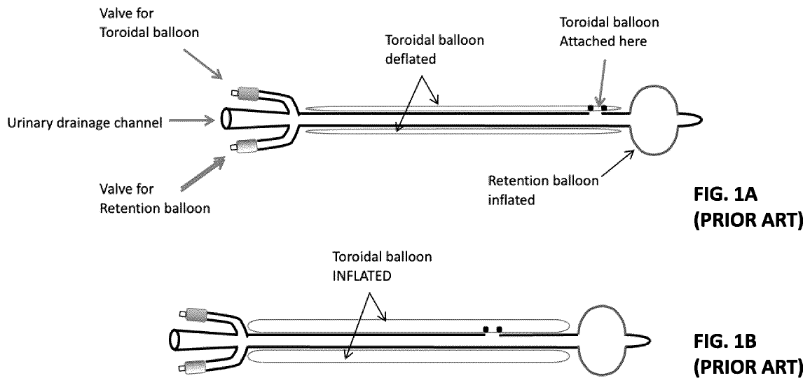

FIG. 1A depicts a diagram of the prior art toroidal foley catheter with a deflated toroidal balloon. FIG. 1B shows a diagram of the prior art toroidal foley catheter with inflated toroidal balloon.

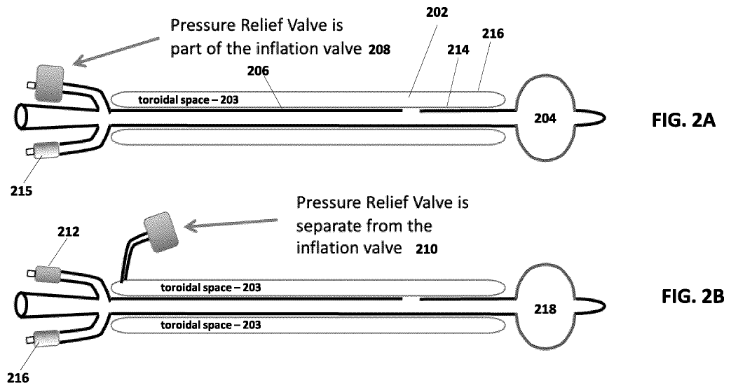

In a first embodiment, as depicted in FIGS. 2A-B, the present invention provides a urethral catheter comprising: at least one toroidal balloon 202 attached over a catheter tube 206, an internal balloon surface 214 of the toroidal balloon(s) 202 contacting itself or contacting itself and the outside of the tube 206, and an external balloon surface 216 of the toroidal balloon 202 configured to contact a urethral wall, the toroidal balloon(s) configured to be deployable in the urethra in a deflated, partially inflated, or fully inflated state, wherein a pressure means is located inside, attached to, or separate from the catheter tube to inflate and/or deflate the toroidal balloon 202. In this embodiment, the catheter tube 206 is moveable along its longitudinal axis inside a toroidal balloon's inner channel, such movement occurring with low friction and reduced sliding of the external balloon surface(s) 216 when contacting the urethral wall. The catheter tube 206 can be extracted by inversion of one or both of the toroidal balloon's internal balloon surface 214 and external balloon surface 216, the inversion enabling low-friction extraction with minimal sliding of the external balloon surface(s) 216 against the urethral wall. Particularly, the internal toroidal balloon surface 214 is lubricated by a lubricious coating, a liquid lubricant, a dry lubricant, or another lubricating means to facilitate the inversion motion. In the continuous embodiment depicted in FIGS. 2A-B, the space enclosed by the internal balloon surface 214 form the toroidal space 203.

In certain embodiments, a lubricious coating or lubricating feature is selectively applied in the toroidal space 203, or to the outside of the catheter shaft at locations where only one side of the toroidal balloon contacts and slides against the catheter tube, or to the internal balloon surface 214 where it contacts itself. This targeted lubricant application reduces friction specifically at the interface between the internal balloon surface(s) and/or the catheter shaft, allowing smooth movement of a portion of the internal toroidal balloon surface 214 relative to the catheter shaft, while substantially limiting or preventing motion of the external balloon surface 216 against the surrounding tissue wall, urethra, or other contacting surfaces.

Materials for achieving lubricity may include inherently lubricious materials for the toroidal balloon and/or the catheter shaft itself. Suitable coating materials include, but are not limited to, polytetrafluoroethylene (PTFE, e.g., Teflon®), polyacrylic acid, hyaluronic acid, or other similarly lubricious materials. Additionally, friction reduction may be achieved by embedding materials such as fabric or molding specific textures directly into the balloon surface. Regardless of the specific method or material chosen, the key consideration is to incorporate lubricious or low-friction characteristics within the internal toroidal space 203 between an internal balloon surface 214 and the catheter shaft 206 to facilitate the desired smooth relative movement.

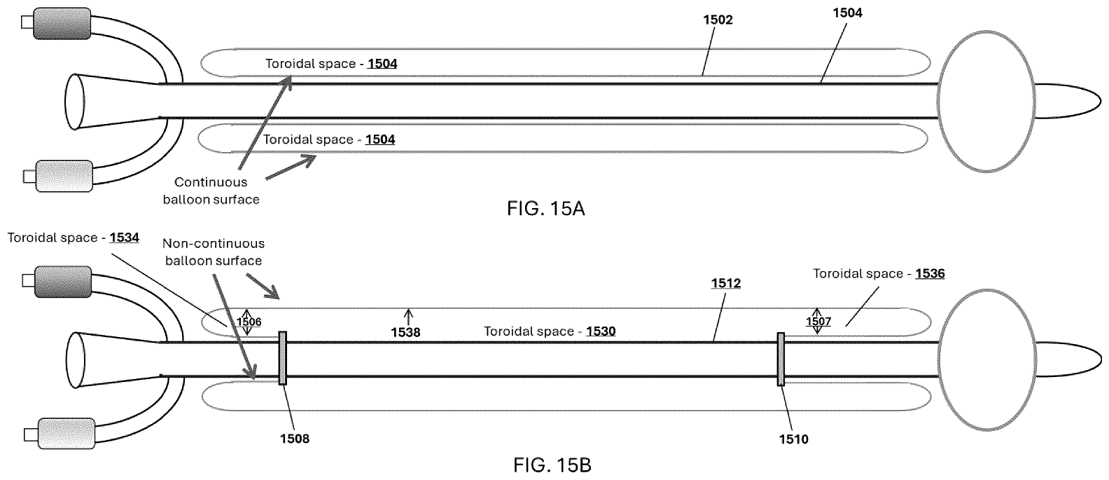

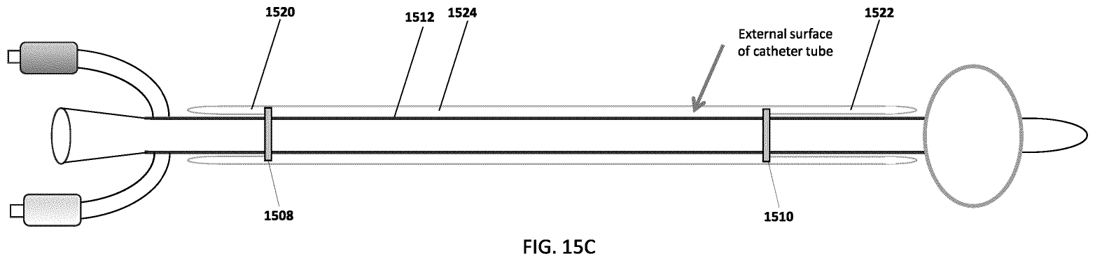

FIGS. 15A-C depict alternate embodiments illustrating various configurations of a toroidal balloon attached to a catheter shaft. FIG. 15A depicts one embodiment which illustrates a continuous balloon surface where the internal balloon surface 1502 is continuous across the catheter shaft 1504. In the continuous embodiment, the internal balloon surface 1502 encloses a toroidal space 1504. FIG. 15B depicts another embodiment illustrating a non-continuous internal balloon surface 1506. In this non-continuous embodiment, the balloon is attached circumferentially to the catheter shaft at points 1508 and 1510, which define a rolling limit (the point where the balloon can no longer roll), and the internal balloon surface 1538 and catheter shaft 1512 create a toroidal space 1530. Optionally, the internal balloon surfaces 1506 and 1507 also create a toroidal space 1534 and 1536, respectively. In the embodiment of FIG. 15B, the balloon surface is not continuous; rather, it is specifically attached to the catheter shaft at designated circumferential attachment points 1508 and 1510. The internal balloon surface is configured to contact either itself or the exterior surface of the catheter tube, as depicted in FIG. 15B. As can be seen in FIG. 15B, a portion of the external surface of catheter tube 1512 does not contact any balloon surface in this embodiment. FIG. 15C depicts the same embodiment in FIG. 15B, but with the balloon deflated. As can be seen in this state, the internal balloon surface and catheter shaft 1512 create a toroidal space 1524. Optionally, the internal surfaces may contact each other at: (1) left of the contact point 1508 to form toroidal space 1520, and (2) to the right of contact point 1510 to form toroidal space 1522.

In the embodiment depicted in FIGS. 15B-C, a lubricant, lubricious coating or lubricious feature is selectively applied in the toroidal space 1534, 1536 and 1520, 1522, or to the external surface of catheter shaft 1512 specifically at locations where only one side of the toroidal balloon contacts and slides against the catheter shaft, or to the internal balloon surface (in toroidal space 1524) where it contacts itself. This targeted lubrication reduces friction at this interface, facilitating smooth relative motion between the internal balloon surface 1506 and the catheter shaft while substantially limiting movement of the external balloon surface against the surrounding tissue wall or urethra.

FIG. 2A depicts an embodiment where the pressure relief valve 208 is part of the closed toroidal balloon system to allow for consistent fill pressure with the balloon, and the pressure relief valve 208 is part of the valve used to inflate the toroidal balloon 202 (as in FIG. 2A), wherein the single unit may be used in both the inflation operation and the in operations of relieving pressure.

In another embodiment, the pressure relief valve 210 is not part of the valve used to inflate and activate the toroidal balloon 202. This embodiment, as depicted in FIG. 2B shows the pressure relief valve 210 as being separate from the inflation valve 212. In FIGS. 2A-2B, elements 215 and 216 are valves used to inflate/deflate the retention balloon 204, 218, respectively. It should be noted that when the catheter is internally deployed, the pressure valve or inflation valves remains external to the patient. In this configuration, a sufficient length of the toroidal balloon extends outside the patient's body (e.g., the urethra), ensuring that any valves are not inadvertently pulled inside.

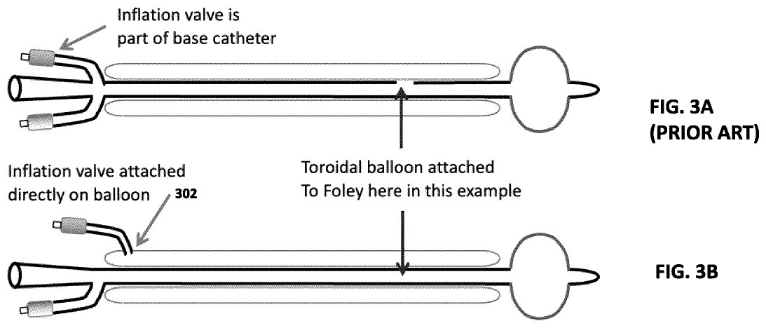

FIG. 3A depicts the prior art toroidal balloon as described in U.S. Pat. No. 10,213,208 wherein the balloon is inflated at the attachment site of the balloon to the catheter. In one embodiment of the present invention, the toroidal balloon is inflated elsewhere on the balloon (as depicted in FIG. 3B), except at the site of attachment of the balloon to the catheter. For example, the inflation valve 302 is shown attached directly to the balloon.

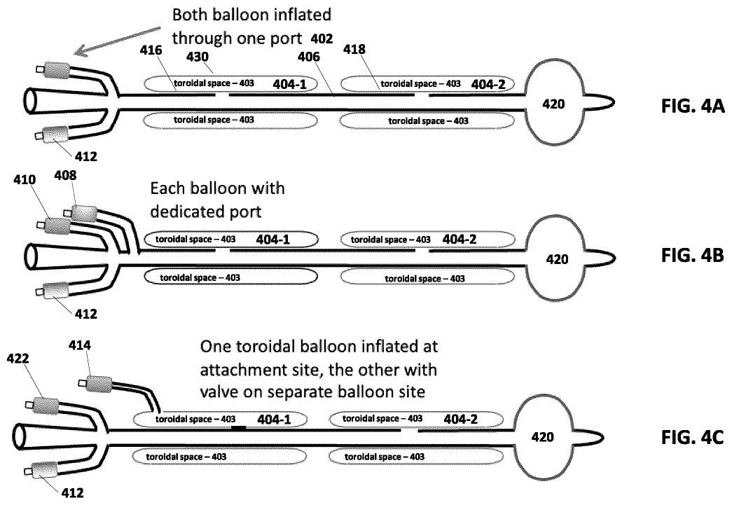

FIGS. 4A-C depict another embodiment of the invention that utilizes a plurality of balloons 404-1 and 404-2. While in this instance, two balloons 404-1 and 404-2 are shown for exemplary purposes only, it should be noted that the number of balloons should not be used to limit the scope of the present invention. In one embodiment described uniquely here, the catheter may also have a plurality of toroidal balloons along the catheter's length that may be inflated via a shared inflation port (as depicted in FIG. 4A), dedicated inflation ports (as depicted in FIG. 4B), or both (as depicted in FIG. 4C). Multiple balloon configurations may be particularly advantageous in situations where the lumen diameter, such as that of the urethra, varies significantly due to strictures or conditions like a swollen prostate. Additionally, multiple balloons can provide targeted pressure adjustment; lower pressures in sensitive areas to enhance patient comfort, and higher pressures at specific sites, such as surgical locations, to optimize therapeutic outcomes. Moreover, partially inflated balloons can be useful for enclosing and subsequently removing foreign objects by deliberately rupturing a balloon. These multiple balloon arrangements would typically be controlled and coordinated manually.

In one embodiment, toroidal balloons may be strategically placed to oppose or roll over certain regions of anatomy such as over the prostate, prostate surgical site, or distal urethra.

FIG. 4A discloses a catheter comprising: (a) an elongated catheter tube 406 defining at least one drainage lumen; (b) a first toroidal balloon 404-1 and a second toroidal balloon 404-2 disposed coaxially around, and axially spaced apart on, the catheter tube 406, each toroidal balloon defining an inner channel through which the catheter tube extends; (c) for each toroidal balloon, an internal balloon surface configured to enclose a toroidal space 403 where it may contact itself (as depicted in embodiments of FIG. 15A) and/or an exterior surface of the catheter tube (as depicted in embodiments of FIGS. 15B-C) and an external balloon surface configured, in use, to contact surrounding tissue; and (d) a pressure-control system comprising at least one inflation valve (402 as depicted in FIG. 4A) fluidly coupled to the toroidal balloons (404-1 and 404-2) and at least one pressure-relief valve (e.g., like pressure relief valve that is part of the inflation valve 208 in FIG. 2A) associated with at least one of the toroidal balloons; wherein the catheter tube 406 is translatable along its longitudinal axis relative to each toroidal balloon, thereby allowing smooth relative movement of the toroidal balloon while substantially reducing friction between the catheter and surrounding tissue; and wherein the internal balloon surface (416, 418) of the first and second toroidal balloons (404-1 and 404-2) carries a lubricant within its respective toroidal space 403, where the lubricant is selected from a hydrophilic coating, a liquid lubricant, a dry lubricant, or combinations thereof. Element 412 in FIGS. 4A-C refers to the inflation port for the retention balloon 420.

In FIG. 4B, the first toroidal balloon 404-1 and the second toroidal balloon 404-2 each have respective dedicated inflation ports (408, 410).

In FIG. 4C, the first toroidal balloon 404-1 includes its own dedicated inflation port (414), while the second toroidal balloon 404-2 is inflated via port 422. It should be noted that any of the toroidal balloons may include its own dedicated port.

In one embodiment, the first toroidal balloon 404-1 is positioned to oppose a patient's prostate, prostate surgical site and the second toroidal balloon 404-2 is positioned to oppose a distal urethral segment.

In one embodiment, the second toroidal balloon 404-2 is configured, upon reaching a rolling limit, to provide a retention function that anchors the catheter in a bladder in the absence of a separate retention balloon.

In one embodiment, at least one of the toroidal balloons (404-1 or 404-2) includes a rupture feature selected from an internal breakaway seam, an external serrated tear line, or a zip thread. In one embodiment, the rupture feature is actuated by a ring 802 carrying a cutting surface 806 positioned to sever the toroidal balloon when that balloon reaches a predetermined rotational position.

In one embodiment, at least a portion of the first toroidal balloon 404-1 overlaps at least a portion of the second toroidal balloon 404-2 when both balloons are inflated.

In one embodiment, the toroidal balloons (404-1 and 404-2) are inserted in a pre-inflated state.

In one embodiment, at least one pressure-relief valve (like 210 in FIG. 2) is mounted directly on the wall of one of the toroidal balloons.

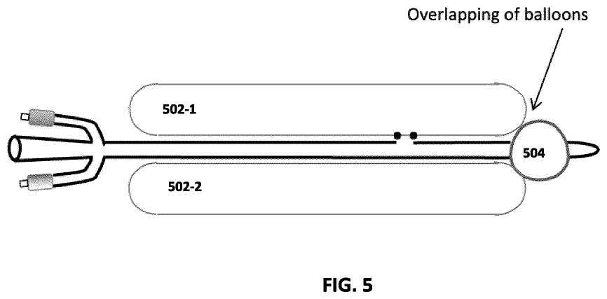

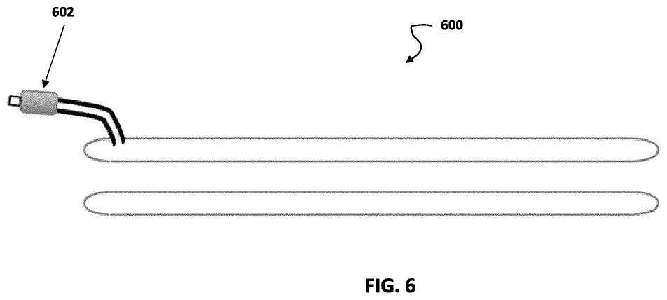

FIG. 5 depicts another embodiment, wherein the toroidal balloons is fashioned to allow overlap with either the retention balloon 504, and either or both of the toroidal balloon portions 502-1 (top portion of the toroidal balloon), 502-2 (bottom portion of the toroidal balloon).

FIG. 6 illustrates an embodiment in which the toroidal balloon 600 has its own inflation port 602 located on the balloon itself. Because the balloon can be inflated at that site, rather than through the catheter's standard inflation lumen, the toroidal balloon functions as a stand-alone device that can be slipped over any off-the-shelf catheter (e.g., a Foley catheter) at the time of surgical deployment.

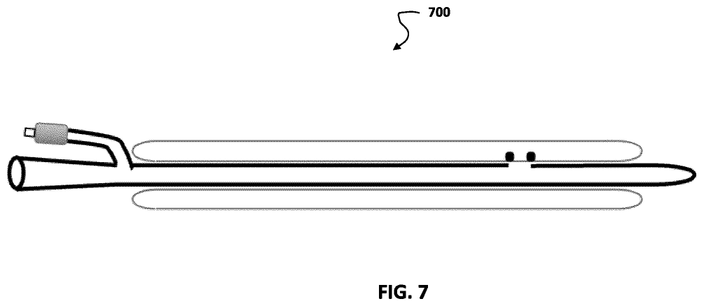

FIG. 7 depicts another embodiment 700 in which the catheter is not equipped with a separate retention balloon the conventional bulb that normally prevents a Foley catheter from slipping out of the bladder. Instead, only the toroidal balloon is present. During normal use the toroidal balloon rolls freely; however, once it reaches its rolling limit, the balloon's geometry creates sufficient resistance against the bladder neck to anchor the catheter in place, thereby performing the retention function on its own.

In one embodiment, the retention balloon is fabricated from a highly flexible material that collapses and folds into the rotating toroidal balloon. By overlapping the toroidal balloon rather than scraping along the urethral wall, the retention balloon functions as a built-in safety feature, protecting the urethra if a patient attempts to remove the catheter without first deflating the balloon.

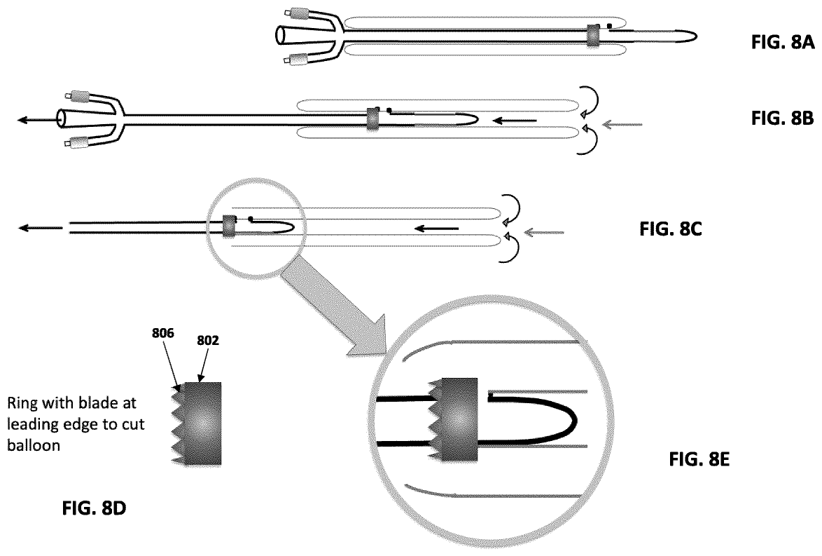

In one embodiment, as depicted in FIGS. 8A-8E, after the retention balloon is deflated and outward tension is applied, the toroidal balloon rolls as the catheter is withdrawn, so its outer surface stays essentially stationary against the urethral wall. When the balloon has rotated far enough that its attachment site reaches the balloon's distal (leading) edge, further rotation stops; from that point on, the balloon's external surface would ordinarily drag across the urethra, causing pain and trauma. To avoid this, the present embodiment deliberately disrupts the balloon exactly when the attachment site reaches the leading edge. A built-in rupture feature, such as an internal breakaway seam, an external serration, or a zip thread, opens the balloon at that moment, allowing the internal proximal fold to continue inverting and slide out as a low-friction sleeve, thereby preserving a rolling, non-dragging extraction path. In the specific configuration shown in FIGS. 8D and 8E, this rupture is produced by a ring 802 carrying a cutting surface 806 positioned at the leading edge; as the balloon reaches the ring, the cutting surface 806 neatly cuts the balloon to initiate the inversion.

It should be noted that the balloon's rolling motion is specifically designed to bring the balloon material into contact with the cutting surface only after the retention balloon has been fully deflated, allowing sufficient catheter withdrawal to engage the cutting surface. As long as the retention balloon remains inflated, it prevents the catheter from moving far enough to trigger the cutting surface. Additionally, the cutting surface is strategically positioned within the balloon but externally relative to the urethra to further prevent any accidental injury to surrounding tissue. The cutting surface itself is designed to cut only through the soft material of the toroidal balloon and is intentionally constructed to be blunt or minimally sharp, eliminating the risk of harming the patient. It may be composed of non-metallic materials for enhanced safety and biocompatibility.

It will be appreciated that, although the present specification describes the invention primarily in the context of a urinary catheter, the inventive toroidal-balloon architecture (and its associated deployment, inversion, and extraction techniques) may likewise be incorporated into other medical catheters, such as vascular, gastrointestinal, or endotracheal tubes, and even into non-medical tubular devices wherever a low-friction, rolling interface is advantageous. Accordingly, the scope of the invention is not limited to the exemplary urinary-catheter implementations set forth herein.

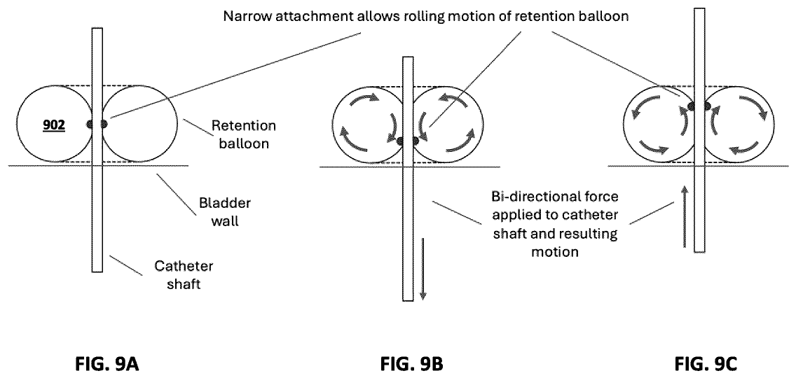

FIGS. 9A-C depict another embodiment, wherein the catheter includes a retention balloon purposely configured to move along the catheter shaft rather than remain fixed, thereby permitting axial motion that existing Foley balloons normally inhibit. By way of example, the retention balloon 902 is secured to the shaft through a very narrow neck or attachment band, allowing the balloon to slide or roll longitudinally relative to the shaft. In this embodiment, the retention balloon is specifically designed to inflate into a “donut” or toroidal shape rather than a traditional spherical shape. Unlike spherical balloons, which have a greater longitudinal distance between their attachment points, the toroidal configuration has closely spaced attachment points along the catheter shaft. This shortened distance between attachment points, combined with the inherent flexibility and normal flexing of the balloon material, naturally permits axial motion along the catheter shaft, thus providing greater and improved patient comfort.

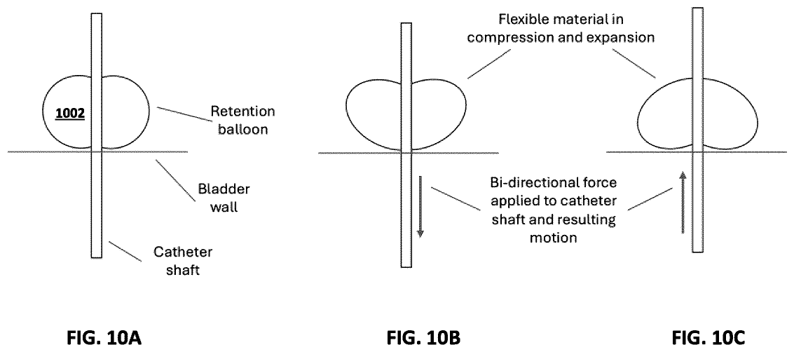

As illustrated in FIGS. 10A-10C, the retention balloon 1002 is mounted so that, when axial force is applied to the catheter shaft, the balloon can roll both inward toward its attachment point and outward away from it. This bidirectional rolling capability allows the balloon to shift along the shaft rather than drag against surrounding tissue, thereby reducing shear forces during insertion and extraction. The rolling or deformation capability of the retention balloon described above is specifically enabled by either a narrow attachment point that naturally encourages the balloon to inflate into a donut-like shape, or by utilizing a particularly soft, flexible balloon material. This soft, pillow-like balloon material easily compresses and deforms under axial pressure, facilitating smooth, low-friction rolling movements along the catheter shaft, thereby further minimizing trauma or discomfort.

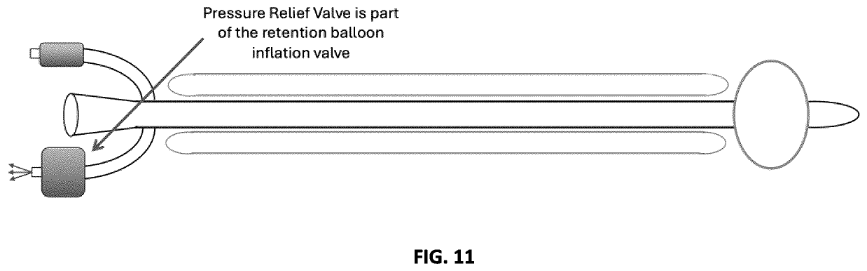

FIG. 11 illustrates an embodiment in which the retention balloon's primary inflation valve also incorporates an over-pressure safety function. The valve body contains a calibrated relief element, such as a spring-loaded poppet or duckbill, set to open automatically when axial traction on the catheter translates into an internal balloon pressure that exceeds a predetermined threshold. Once that threshold is reached, the valve vents the inflation lumen, deflating the balloon and allowing the catheter to exit with minimal urethral trauma. Normal inflation and deflation are performed through the same valve port; the safety feature activates only under excessive pull force.

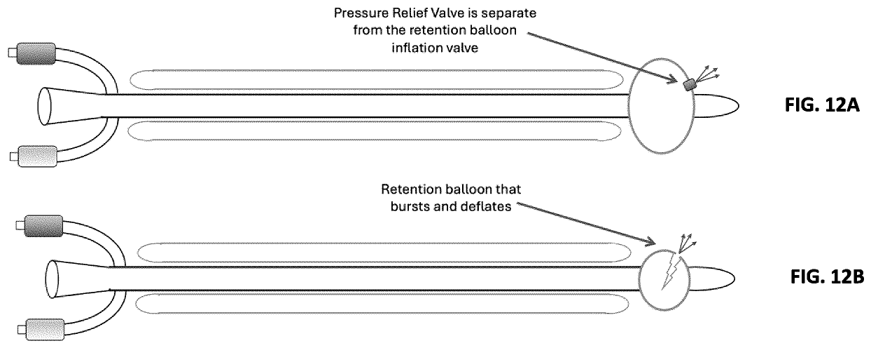

FIG. 12A depicts a variant in which the retention balloon has two fluid paths: a standard inflation/deflation valve and an independent pressure-relief valve located elsewhere on the balloon circuit. During routine use the balloon is filled or emptied exclusively through the main valve. If the catheter is inadvertently pulled while the balloon remains inflated, the separate relief valve senses the resulting pressure spike and opens, rapidly venting the balloon. Physically isolating the safety valve from the main port simplifies calibration and allows the relief component to be factory-sealed and tamper-resistant.

FIG. 12B shows an alternative safety strategy that eliminates auxiliary valves altogether. Here, the balloon wall is engineered with a deliberate weak zone (e.g., a scored seam, thinned annulus, or embedded tear strip) that ruptures when traction on the catheter raises internal pressure beyond a preset limit. The instant the seam yields, the balloon collapses and deflates, converting the inflated cuff into a limp membrane that slides out with minimal resistance. This single-use “burst-deflate” approach provides a fail-safe exit path without the complexity of moving valve parts.

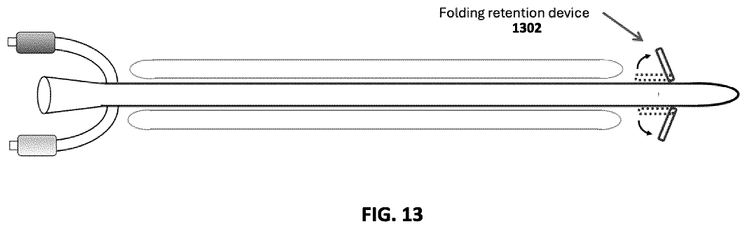

FIG. 13 illustrates an embodiment in which the catheter carries a flexible, non-balloon retention device 1302 (e.g., a flange or umbrella—that lies folded flat against the shaft for insertion). Once the assembly reaches the bladder, device 1302 automatically unfolds, creating an anchoring surface that prevents inadvertent catheter withdrawal.

The same retention device 1302 is also engineered to respond safely to unintended extraction. If traction is applied before deliberate removal, element 1302 folds forward, collapsing into the toroidal balloon's central channel. As the toroidal balloon inverts during withdrawal, it envelops the folded retention member, forming a smooth, low-friction sleeve that lets the catheter slide out with minimal discomfort or tissue trauma.

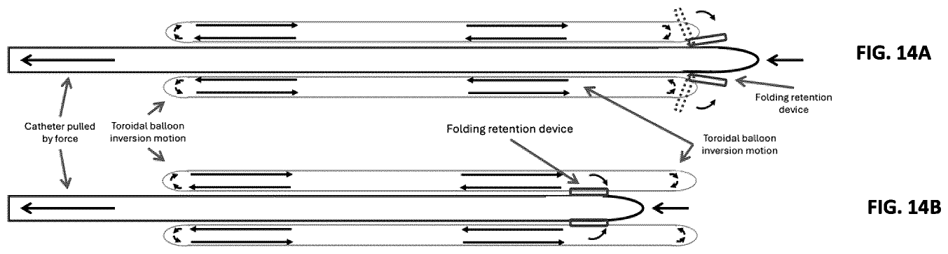

FIGS. 14A and 14B depict an embodiment in which the catheter's retention device is not an inflatable bulb; instead, it is a mechanically folding element that lies flat for insertion and then opens inside the bladder to hold the catheter in place. During withdrawal the element folds forward and is enveloped by the rotating toroidal balloon, which inverts to create a smooth sleeve and allows low-friction extraction. The specification further contemplates alternative retention geometries, such as irregularly shaped inflatable cuffs or other mechanical anchors, that would likewise be shielded by the toroidal balloon as the catheter is retracted.

In another embodiment, both a lubricant and an antibacterial agent are incorporated into the toroidal-balloon system yet remain spatially separated so neither dilutes nor counteracts the other. For example, the lubricant may reside within the inner toroidal channel while the antibacterial agent coats the outward-facing surface of the balloon or sleeve. Either or both substances may be applied as surface coatings or infused directly into the balloon or catheter materials.

In a further embodiment, the toroidal balloon is inserted either completely uninflated or already pre-inflated, in contrast to the preceding embodiments in which the catheter is placed first and the balloon is inflated only after proper positioning.

Various modifications to these aspects will be readily apparent, and the generic principles defined herein may be applied to other aspects. Thus, the claims are not intended to be limited to the aspects shown herein, but is to be accorded the full scope consistent with the language claims, where reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject technology.

A phrase, for example, an “aspect” does not imply that the aspect is essential to the subject technology or that the aspect applies to all configurations of the subject technology. A disclosure relating to an aspect may apply to all configurations, or one or more configurations. A phrase, for example, an aspect may refer to one or more aspects and vice versa. A phrase, for example, a “configuration” does not imply that such configuration is essential to the subject technology or that such configuration applies to all configurations of the subject technology. A disclosure relating to a configuration may apply to all configurations, or one or more configurations. A phrase, for example, a configuration may refer to one or more configurations and vice versa.

The various embodiments described above are provided by way of illustration only and should not be construed to limit the scope of the disclosure. Those skilled in the art will readily recognize various modifications and changes that may be made to the principles described herein without following the example embodiments and applications illustrated and described herein, and without departing from the spirit and scope of the disclosure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results.

As noted above, particular embodiments of the subject matter have been described, but other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

### CONCLUSION

A system and method have been disclosed for effectively implementing rolling toroidal balloon catheter systems, which optionally include multi-balloon configurations, internal friction-reducing features, low-impact removal, and safety release mechanisms. While various preferred embodiments have been shown and described, it will be understood that there is no intent to limit the invention by such disclosure, but rather, it is intended to cover all modifications falling within the spirit and scope of the invention, as defined in the appended claims.

What is claimed is:

1. A catheter comprising:
a) an elongated catheter shaft defining at least one drainage lumen;
b) at least one toroidal balloon disposed coaxially around the catheter shaft and having an internal balloon surface, the internal balloon surface being either:
i. continuous across the catheter shaft, thereby defining an inner channel through which the catheter shaft extends, the internal balloon surface 6 enclosing a first toroidal space; or
ii. non-continuous across the catheter shaft, the internal balloon surface having circumferential first and second attachment points to the catheter shaft, such that the internal balloon surface together with the catheter shaft bounds a second toroidal space;
c) an external balloon surface configured, in use, to contact a body lumen wall; and
d) a lubricant selectively applied to (i) the catheter shaft, (ii) the internal balloon surface, (iii) the first toroidal space when the internal balloon surface is continuous, or (iv) the second toroidal space when the internal balloon surface is non-continuous, the lubricant reducing friction at regions where opposed portions of the internal balloon surface contact each other and at regions where the internal balloon surface contacts the catheter shaft, thereby allowing smooth relative movement of the toroidal balloon while substantially limiting motion of the external balloon surface against surrounding tissue.

2. The catheter of claim 1, wherein, in addition to the second toroidal space, the internal balloon surfaces form a third toroidal space circumferentially adjacent to the first attachment point; and the internal balloon surfaces form a fourth toroidal space circumferentially adjacent to the second attachment point.

3. The catheter of claim 1, wherein the lubricant is selected from the group consisting of a lubricious coating such as a hydrophilic coating, a liquid lubricant, a dry lubricant, an inherently lubricious material, an embedded fabric, a textured surface molded directly into the balloon surface, and combinations thereof.

4. The catheter of claim 1, wherein the catheter shaft is translatable along its longitudinal axis relative to the toroidal balloon, such that, during catheter withdrawal, at least a portion of the toroidal balloon inverts, thereby reducing friction between the catheter and the body lumen wall.

5. The catheter of claim 1, wherein the catheter further comprises a pressure-control system, the pressure-control system comprising:
a) an inflation/deflation valve fluidly coupled to the toroidal balloon, and
b) a relief valve mounted on the wall of the toroidal balloon and physically separate from the inflation/deflation valve, wherein the relief valve is configured to vent the balloon at a preset pressure.

6. The catheter of claim 4, wherein the inflation/deflation valve further includes an integral pressure-relief element in addition to the relief valve.

7. The catheter of claim 1, wherein the toroidal balloon includes an inflation port disposed directly on the balloon wall at a location spaced from the balloon's attachment region to the catheter shaft.

8. The catheter of claim 1, wherein the toroidal balloon is configured, upon reaching a rolling limit, to provide a retention function that anchors the catheter in a bladder in the absence of a separate retention balloon.

9. The catheter of claim 1, wherein the toroidal balloon includes a rupture feature selected from an internal breakaway seam, an external serrated tear line, or a zip thread.

10. The catheter of claim 9, wherein the rupture feature is actuated by a ring carrying a cutting surface positioned to sever the toroidal balloon when the balloon reaches a predetermined rotational position.

11. The catheter of claim 1, wherein the catheter further comprises a retention balloon, wherein the toroidal balloon overlaps a portion of the retention balloon when both are inflated.

12. The catheter of claim 1, wherein the toroidal balloon is inserted in a pre-inflated state.

13. The catheter of claim 1, wherein the toroidal balloon is positioned along the catheter tube to oppose a predetermined anatomical region selected from the group consisting of the prostate, a surgical side associated with the prostate, and the distal urethra.

14. The catheter of claim 1, further comprising a retention balloon disposed coaxially around the catheter shaft, wherein the retention balloon is configured to inflate into a toroidal shape and is secured to the catheter shaft by closely spaced circumferential attachment points forming a narrow neck or attachment band, thereby permitting axial sliding or rolling motion of the retention balloon longitudinally along the catheter shaft.

15. The catheter of claim 1, further comprising a retention balloon coaxially mounted around the catheter shaft, wherein the retention balloon is secured to the catheter shaft by a narrow 2 attachment point or neck, and wherein the retention balloon is formed from a soft, flexible material configured to roll bidirectionally inward toward, and outward away from, the attachment point when axial force is applied to the catheter shaft, thereby facilitating axial motion of the catheter shaft and reducing shear forces and tissue trauma.

16. A catheter comprising:
a) an elongated catheter shaft defining at least one drainage lumen;
b) first and second toroidal balloons disposed coaxially around the catheter shaft and axially spaced apart from one another, each of the first and second toroidal balloons including
i. an internal balloon surface that is either:
1. continuous across the catheter shaft, thereby defining an inner channel through which the catheter shaft extends and the internal balloon surface encloses a first toroidal space; or
2. non-continuous across the catheter shaft, the internal balloon surface having circumferential first and second attachment points to the catheter shaft, such that the internal balloon surface together with the catheter shaft bounds a second toroidal space;
ii. an external balloon surface configured, in use, to contact a body lumen wall; and
iii. a lubricant selectively applied to (A) the catheter shaft, (B) the internal balloon surface, (C) the first toroidal space when the internal balloon surface is continuous, or (D) the second toroidal space when the internal balloon surface is non-continuous, the lubricant reducing friction at regions where opposed portions of the internal balloon surface contact each other and at regions where the internal balloon surface contacts the catheter shaft, thereby allowing smooth relative movement of each toroidal balloon while substantially limiting motion of the external balloon surface against surrounding tissue.

17. The catheter of claim 16, wherein the catheter further comprises a pressure-control system comprising at least one inflation valve fluidly coupled to the toroidal balloons and at least one pressure-relief valve associated with at least one of the toroidal balloons.

18. The catheter of claim 16, wherein the first toroidal balloon and the second toroidal balloon each have respective dedicated inflation ports.

19. The catheter of claim 16, wherein either the first toroidal balloon or second toroidal balloon includes its own dedicated inflation port.

20. The catheter of claim 16, wherein the first toroidal balloon is positioned to oppose a patient's prostate or a surgical site near a patient's prostate and the second toroidal balloon is positioned to oppose a distal urethral segment.

21. The catheter of claim 16, wherein the second toroidal balloon is configured, upon reaching a rolling limit, to provide a retention function that anchors the catheter in a bladder in the absence of a separate retention balloon.

22. The catheter of claim 16, wherein at least one of the toroidal balloons includes a rupture feature selected from an internal breakaway seam, an external serrated tear line, or a zip thread.

23. The catheter of claim 22, wherein the rupture feature is actuated by a ring carrying a cutting surface positioned to sever the toroidal balloon when that balloon reaches a predetermined 2 rotational position.

24. The catheter of claim 16, wherein the catheter further comprises a retention balloon, wherein at least a portion of at least one of the toroidal balloons overlaps a portion of the retention balloon or a portion of the retention balloon overlaps a portion of at least one of the toroidal balloons when both are inflated.

25. The catheter of claim 16, wherein the toroidal balloons are inserted in a pre-inflated state.

26. The catheter of claim 16, wherein at least one pressure-relief valve is mounted directly on the wall of one of the toroidal balloons.
